(19) United States
(12) Patent Application Publication
(10) Pub. No.: US 2020/0330707 A1
(43) Pub. Date: Oct. 22, 2020

(54) NOSE CANNULA COVER SYSTEMS AND METHOD

(71) Applicant: William Connor Ellington, IV, BELLEVIEW, FL (US)

(72) Inventor: William Connor Ellington, IV, BELLEVIEW, FL (US)

(21) Appl. No.: 16/549,951

(22) Filed: Aug. 23, 2019

Related U.S. Application Data
(60) Provisional application No. 62/835,824, filed on Apr. 18, 2019.

(51) Int. Cl. A61M 16/00; A61M 16/06

(52) U.S. Cl. CPC A61M 16/0003; A61M 2209/06; A61M 16/0666

(57) ABSTRACT

A protective case for a nose cannula when not in use. It effectively ensures that the device is protected from dirt, dust, and bacteria. It prevents individuals from placing a contaminated cannula back into their nose. It keeps dirt or bacteria from causing an infection by entering the nasal passage and lungs. It offers a convenient and accessible cover with an optional decorative design.

### CROSS-REFERENCE TO RELATED APPLICATION(S)

The present application is related to and claims priority to U.S. Provisional Patent Application No. 62/835,824 filed Apr. 18, 2019, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### Technical Field

The present invention relates generally to the field of storage containers of existing art and more specifically relates to storage means for a nose cannula.

### Related Art

Many people wear a nose cannula. When a portable nose cannula is taken out of a user's nose for eating or other purposes, the cannula is often set onto the nearest surface, such as a bed, floor, or table. This can result in the cannula becoming contaminated with dirt, debris, and bacteria. When the user places the device back into their nose, these contaminants may travel through the nasal passage to the lungs. This can lead to serious health issues. A suitable solution is desired.

U.S. Pub. No. 2009/0292258 to Charles Turner relates to a nasal cannula cover. The described nasal cannula cover is a protective cover for a nasal cannula. The device completely encloses the two prongs of the nasal cannula when properly installed. This protective cover will keep the nasal cannula from contacting potentially unclean surfaces when the nasal cannula is not in use.

### SUMMARY OF THE INVENTION

In view of the foregoing disadvantages inherent in the known storage containers art, the present disclosure provides a novel nose cannula cover system. The general purpose of the present disclosure, which will be described subsequently in greater detail, is to provide an efficient and effective nose cannula cover system.

The present invention is a protective case for a nose cannula when the nose cannula is not in use. It effectively ensures that the stored device is protected from dirt, dust, and bacteria. It helps prevent individuals from placing a contaminated cannula back into their nose. It keeps dirt or bacteria from causing an infection by entering the nasal passage and lungs. It offers a convenient and accessible cover with an optional decorative design. The present invention completely isolates the two prongs of the nasal cannula when used and leaves them sitting in a favorable positioning for re-use (forward-facing).

A nose cannula cover system is disclosed herein, the system comprising: a nose cannula case having a body with a top-shell; a bottom-shell; a first-supportive-liner; a second-supportive-liner; and a thickness; wherein the body is defined by the top-shell hingedly related to the bottom-shell, the first-supportive-liner nested in the top-shell and the second-supportive-liner nested in the bottom-shell, together forming the thickness when in a closed-condition. The first-supportive-liner and the second-supportive-liner provide a contoured inner volume (full supporting surface such as not to damage the nose cannula during storage), when mated, such that the nasal cannula and attached hosing (tubing) are able to be substantially contained therein for sanitary storage, the hosing not pinched but held in stasis by the nose cannula case with hosing protruding from each respective side.

A method of use for the nose cannula cover system is also disclosed herein, the method comprising the steps of: providing a nose cannula case (having a body with a top-shell; a bottom-shell; a first-supportive-liner; a second-supportive-liner; and a thickness); nesting a nasal cannula within a contoured inner volume of the first-supportive-liner and the second-supportive-liner such that the nose cannula is fully supporting along its whole length and not compromised by pinching or uneven surface pressure upwardly); placing hosing attached to the nasal cannula through left and right receivers; and closing the nose cannula case about the connected nasal cannula and hosing (assembly) to isolate the nasal cannula from external ambient elements such as dirt, debris, contacted bacteria, etc. The method may further comprise the step of: opening the nose cannula case to access and use the nasal cannula (assembly) in a ready-to-use condition (best positioned for next re-use) as attached to the hosing.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a nose cannula cover system, constructed and operative according to the teachings of the present disclosure.

FIG. 1 is a perspective view of the nose cannula cover system, according to an embodiment of the disclosure.

FIG. 2 is a perspective view of the nose cannula cover system of FIG. 1, as carried, according to an embodiment of the present disclosure.

FIG. 3 is a perspective view of the nose cannula cover system of FIG. 1, according to an embodiment of the present disclosure.

FIG. 4 is a perspective view of the nose cannula cover system of FIG. 1, the nose cannula as seated (prongs forward-facing), according to an embodiment of the present disclosure.

FIG. 5 is a perspective view of the nose cannula cover system of FIG. 1, the nose cannula system as attached to a carabiner, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a storage container means and more particularly to a nose cannula cover system as used to improve the protection of a nose cannula and preventing contamination of the device.

Generally, the Nose Cannula Cover is a case designed to hold a nose cannula when it is not in use and is ready for the next use. The present invention is preferably comprised of a protective case with a hinge and a front clasp. The case has two side openings (soft edges) to allow the hosing to be connected (as an assembly) while covering the nasal cannula. The interior of the device may include a (fully) supportive molded lining where the cannula can be placed. The device has an eyelet on the backside by the hinge to connect it to a carabiner. The device may be available in a variety of colors and decorative 3D designs.

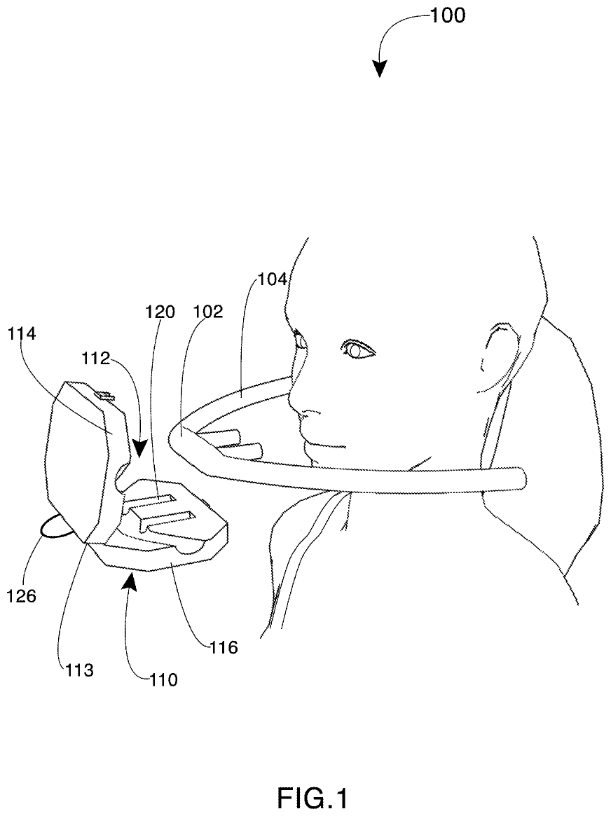

Referring now more specifically to the drawings by numerals of reference, there is shown in FIGS. 1-5, various views of a nose cannula cover system 100.

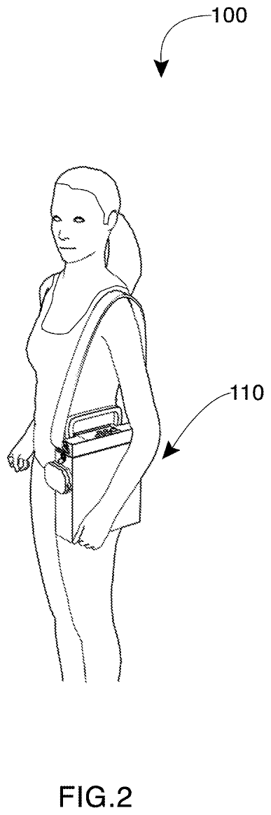

FIG. 1 shows a nose cannula cover system 100, according to an embodiment of the present disclosure. Here, the nose cannula cover system 100 may be beneficial for use by a user to safely store the nose cannula 102 and to limit contamination of the nose cannula 102. As illustrated, the nose cannula cover system 100 may include a nose cannula case 110 having a body 112 with a top-shell 114; a bottom-shell 116; a first-supportive-liner 115; a second-supportive-liner 117; and a thickness 118. The body 112 is defined by the top-shell 114 hingedly related to the bottom-shell 116, the first-supportive-liner 115 nested in the top-shell 114 and the second-supportive-liner 117 nested in the bottom-shell 116, together forming the thickness 118 when in a closed-condition. The first-supportive-liner 115 and the second-supportive-liner 117 provide a contoured inner volume 120 (full supporting surface such as not to damage the nose cannula 102 during storage), when mated, such that the nasal cannula 102 and attached hosing 104 are able to be substantially contained therein for sanitary storage, the hosing 104 not pinched but held in stasis by the nose cannula case 110 with hosing 104 protruding from each respective side. FIG. 1 shows how the nose cannula 102 is removed and placed into the nose cannula case 110. FIG. 2 shows the nose cannula case 110 being carried by the user.

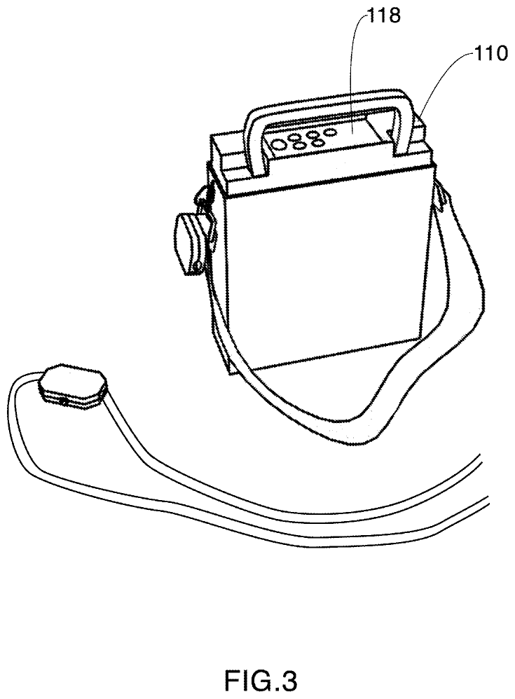

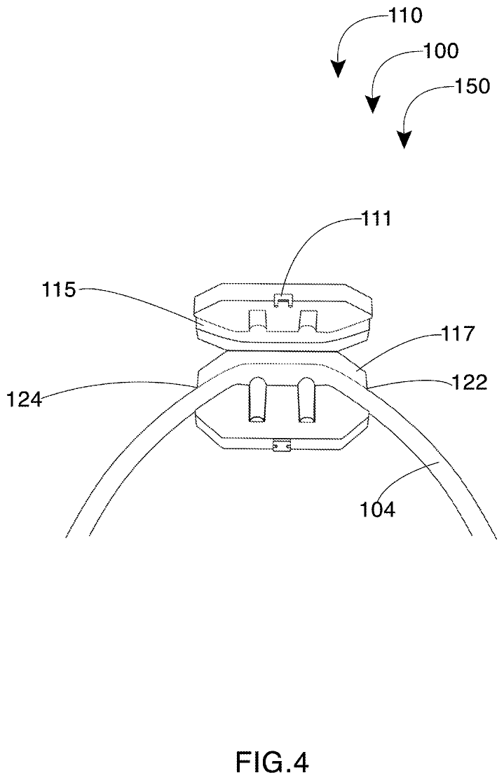

FIG. 3 shows how the hosing 104 (tubing) is able to run through the nose cannula case 110 and FIG. 4 shows the nose cannula 102 in place in a ready for in-use condition 150. In preferred embodiments the nose cannula cover system 100 comprises at least one hinge 113 preferably located on the back of the nose cannula case 110, as shown. Further, the present invention may comprise a front-securer 111. The first-supportive-liner 115 and the second-supportive-liner 117 are molded each as a one-piece solid construction such that the nose cannula 102 and hosing 104 are supported along the entire length of the ‘held portion’ such that support is firm and consistent along the entire length. Without this consistent support the nose cannula 102 and hosing 104 may become damaged which is undesirable. The present invention provides that the two prongs of the nasal cannula are forward-facing for next use.

The first-supportive-liner 115 and the second-supportive-liner 117 preferably comprise soft, pliable yet supportive material (foam or the like); wherein the first-supportive-liner 115 and the second-supportive-liner 117 align the nose cannula 102 in a forward-facing static positioning relative the nose cannula case 110 for ease of access as shown. The hosing 104 passes through a right-receiver 122 when the nose cannula case 110 is in the closed-condition and in a similar way the hosing 104 passes through a left-receiver 124. The nasal cannula 102 when stored in the nose cannula case 110 during the closed-condition is isolated from external ambient dirt, debris and bacteria. The right-receiver 122 comprises a soft-edge and the left-receiver 124 comprises a soft-edge. The right-receiver 122 is also circular as is the left-receiver 124 is circular. The first-supportive-liner 115 and the second-supportive-liner support 117 hold the nose cannula 102 in a slight friction-fit during the sanitary storage.

According to one embodiment, the nose cannula cover system 100 may be arranged as a kit. In particular, the nose cannula cover system 100 may further include a set of instructions. The instructions may detail functional relationships in relation to the structure of the nose cannula cover system 100 such that the nose cannula cover system 100 can be used, maintained, or the like, in a preferred manner. When used, the device also prevents unwanted damage to the nasal cannula 102.

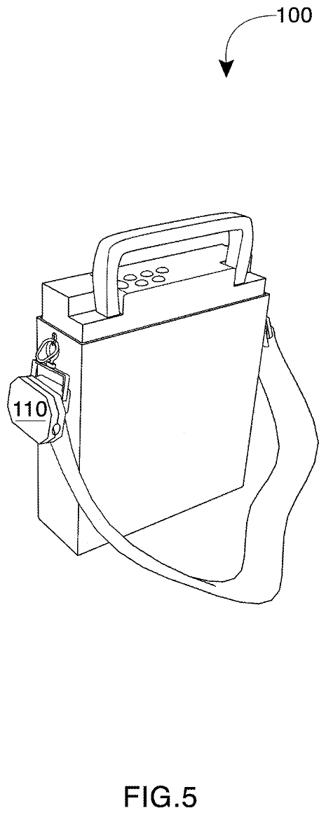

FIG. 5 shows the nose cannula case 110 attached to a carabiner. The nose cannula case 110 comprises an eyelet 126, as shown in FIG. 5. The eyelet 126 allows for attachment to a clip (fastener such as a carabiner) for hands-free carrying of the nose cannula case 110 during transport. Upon reading this specification, it should be appreciated that, under appropriate circumstances, considering such issues as user preferences, design preference, structural requirements, marketing preferences, cost, available materials, technological advances, etc., other fastening means and arrangements such as, for example, clips, clasps, etc., may be sufficient.

A method for use of the nose cannula cover system 100 may include the steps of: providing a nose cannula case (having a body with a top-shell; a bottom-shell; a first-supportive-liner; a second-supportive-liner; and a thickness) as described above; nesting a nasal cannula within a contoured inner volume of the first-supportive-liner and the second-supportive-liner such that the nose cannula is fully supporting along its whole length and not compromised by pinching or uneven surface pressure upwardly); placing hosing attached to the nasal cannula through left and right receivers; and closing the nose cannula case about the connected nasal cannula and hosing (assembly) to isolate the nasal cannula from external ambient elements such as dirt, debris, contacted bacteria, etc. The method may further comprise the step of: opening the nose cannula case to access and use the nasal cannula (assembly) in a ready-to-use condition as attached to the hosing. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for use, are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A nose cannula cover system, the system comprising:
a nose cannula case having a body with
a top-shell;
a bottom-shell;
a first-supportive-liner;
a second-supportive-liner; and
a thickness;
wherein said body is defined by said top-shell hingedly related to said bottom-shell, said first-supportive-liner nested in said top-shell and said second-supportive-liner nested in said bottom-shell, together forming said thickness when in a closed-condition; and
wherein said first-supportive-liner and said second-supportive-liner provide a contoured inner volume, when mated, such that a nasal cannula and attached hosing are able to be substantially contained therein for sanitary storage, said hosing not pinched but held in stasis by said nose cannula case.

2. The nose cannula cover system of claim 1, further comprising at least one hinge.

3. The nose cannula cover system of claim 2, further comprising a front-securer.

4. The nose cannula cover system of claim 3, wherein said first-supportive-liner and said second-supportive-liner are molded each as a one-piece solid construction.

5. The nose cannula cover system of claim 4, wherein said first-supportive-liner and said second-supportive-liner comprise soft, pliable material.

6. The nose cannula cover system of claim 5, wherein said first-supportive-liner and said second-supportive-liner align said nose cannula in a forward-facing static positioning.

7. The nose cannula cover system of claim 6, wherein the hosing passes through a right-receiver when said nose cannula case is in said closed-condition.

8. The nose cannula cover system of claim 7, wherein the hosing passes through a left-receiver when in said nose cannula case is in said closed-condition.

9. The nose cannula cover system of claim 8, wherein the nasal cannula when stored in said nose cannula case during said closed-condition is isolated from external ambient dirt, debris and bacteria.

10. The nose cannula cover system of claim 9, wherein the right-receiver comprises a soft-edge.

11. The nose cannula cover system of claim 9, wherein the left-receiver comprises a soft-edge.

12. The nose cannula cover system of claim 10, wherein the right-receiver is circular.

13. The nose cannula cover system of claim 11, wherein the left-receiver is circular.

14. The nose cannula cover system of claim 11, wherein the nose cannula case comprises an eyelet.

15. The nose cannula cover system of claim 14, wherein the eyelet allows for attachment to a clip for hands-free carrying of said nose cannula case during transport.

16. The nose cannula cover system of claim 6, wherein said first-supportive-liner and said second-supportive-liner support and hold said nose cannula in a slight friction-fit during said sanitary storage.

17. A nose cannula cover system, the system comprising:
a nose cannula case having a body with
a top-shell;
a bottom-shell;
a first-supportive-liner;
a second-supportive-liner;
at least one hinge;
a front-securer;
an eyelet; and
a thickness;
wherein said body is defined by said top-shell hingedly related to said bottom-shell, said first-supportive-liner nested in said top-shell and said second-supportive-liner nested in said bottom-shell, together forming said thickness when in a closed-condition;
wherein said first-supportive-liner and said second-supportive-liner provide a contoured inner volume, when mated, such that a nasal cannula and attached hosing are able to be substantially contained therein for sanitary storage, said hosing not pinched but held in stasis by said nose cannula case;
wherein said first-supportive-liner and said second-supportive-liner are molded;
wherein said first-supportive-liner and said second-supportive-liner comprise soft, pliable material;
wherein said first-supportive-liner and said second-supportive-liner align said nose cannula in a forward-facing static positioning when used;
wherein the hosing passes through a right-receiver and a left-receiver when said nose cannula case is in said closed-condition;
wherein the nasal cannula when stored in said nose cannula case during said closed-condition is isolated from external ambient dirt, debris and bacteria;
wherein the right-receiver and said left-receiver each comprise a soft-edge and are circular when mated; and
wherein the eyelet allows for attachment to a carabiner-clip for hands-free carrying of said nose cannula case during transport.

18. The nose cannula cover system of claim 17, further comprising set of instructions; and
wherein the nose cannula cover system is arranged as a kit.

19. A method of use for the nose cannula cover system, the method comprising the steps of:
providing a nose cannula case having a body with
a top-shell;
a bottom-shell;
a first-supportive-liner;
a second-supportive-liner; and
a thickness;
nesting a nasal cannula within a contoured inner volume of said first-supportive-liner and said second-supportive-liner;
placing hosing attached to said nasal cannula through left and right receivers; and
closing said nose cannula case about connected said nasal cannula and said hosing to isolate said nasal cannula from external ambient elements.

20. The method of claim 19, further comprising the step of:
opening said nose cannula case to access and use said nasal cannula in a ready-to-use condition as attached to said hosing.
